(19) United States
(12) Patent Application Publication
(10) Pub. No.: US 2006/0100496 A1
(43) Pub. Date: May 11, 2006

(54) Device and method for in vivo illumination

(76) Inventor: Jerome Avron, Haifa (IL)

Correspondence Address: PEARL COHEN ZEDEK, LLP, 1500 BROADWAY 12TH FLOOR, NEW YORK, NY, 10036, US

(21) Appl. No.: 10/974,979

(22) Filed: Oct. 28, 2004

(51) Int. Cl. A61B 5/05

(52) U.S. Cl. 600/407

(57) ABSTRACT

An in vivo imaging device including a hybrid illumination unit. The hybrid illumination unit may include, for example, a plurality of discrete light sources and/or resistors and/or optical resin.

### FIELD OF THE INVENTION

The present invention relates to a device useful for in-vivo imaging, more specifically to a device for providing illumination in-vivo.

### BACKGROUND OF THE INVENTION

Known devices may be helpful in providing in-vivo imaging. Autonomous in-vivo imaging devices, such as swallowable or ingestible capsules or other devices may move through a body lumen, imaging as they move along. In vivo imaging may require in-vivo illumination, for example, using one or more light sources for example Light Emitting Diodes (LEDs) or other suitable sources positioned inside an in-vivo imaging device.

In some in vivo devices, such as ingestible imaging capsules, the electronic components within the capsule, such as light sources, may be arranged on a board or on several boards, for example on a printed circuit board (PCB). In some cases proper alignment or positioning of components, such as light sources, may be difficult to achieve.

### SUMMARY OF THE INVENTION

Thus the present invention provides, according to some embodiments, an in vivo device such as an imaging device including an illumination sub system, such as a hybrid illumination unit. According to one embodiment the hybrid illumination unit may include, for example, a substrate or support, such as a PCB, for holding one or more light sources, for example, LEDs or other suitable light sources.

The need for an illumination unit, for example a hybrid illumination unit stems from the growing demand for an in vivo device characterized by a high level of detail and finish which enables exact and powerful illumination in accordance with the highly specific demands of the in vivo device. Also there is a need for an in vivo device that has already been calibrated and fitted, for example with the necessary illumination unit prior to it's insertion into the in vivo device. Thus, according to one embodiment of the invention, a pre-calibrated and arranged hybrid illumination unit may fit into the in vivo device, for example a swallowable capsule, so highly expensive and time consuming additional production steps are not necessary.

According to one embodiment, the hybrid illumination unit may include at least a resistor in order to set different levels of illumination.

In another embodiment a plurality of discrete light sources may be mounted on the hybrid illumination unit in order to direct and focus illumination as required by the in vivo device or operation performed.

In another embodiment, the hybrid illumination unit may be mounted on a circuit board, such as a flexible PCB, which is folded and inserted to an in vivo device.

In yet another embodiment of the present invention, the hybrid illumination unit may be manufactured according to several designs, enabling the support to fit into in vivo devices of different shapes.

### BRIEF DESCRIPTION OF THE DRAWINGS

The principles and operation of the system, apparatus, and method according to the present invention may be better understood with reference to the drawings, and the following description, it being understood that these drawings are given for illustrative purposes only and are not meant to be limiting, wherein:

FIG. 1 shows a schematic illustration of an in-vivo imaging device, according to one embodiment of the invention;

FIG. 2A-2C shows a schematic illustration of a hybrid illumination unit, according to one embodiment of the invention;

FIG. 3A-3B shows a schematic illustration of a circuit board, according to one embodiment of the invention;

FIG. 4 is a flowchart depicting a method for producing a hybrid illumination unit, according to embodiments of the invention;

Fig. 5 is a flowchart depicting a method for producing an in vivo device which includes a hybrid illumination unit, according to embodiments of the invention; and

Fig. 6 is a flowchart depicting a method for in vivo imaging, according to embodiments of the invention.

It should be noted that for simplicity and clarity of illustration, elements shown in the figures have not necessarily been drawn to scale. For example, the dimensions of some of the elements may be exaggerated relative to other elements for clarity. Furthermore, where considered appropriate, reference numerals may be repeated among the figures to indicate corresponding or analogous elements throughout the serial views.

### DETAILED DESCRIPTION OF THE INVENTION

The following description is presented to enable one of ordinary skill in the art to make and use the invention as provided in the context of a particular application and its requirements. Various modifications to the described embodiments will be apparent to those with skill in the art, and the general principles defined herein may be applied to other embodiments. Therefore, the present invention is not intended to be limited to the particular embodiments shown and described, but is to be accorded the widest scope consistent with the principles and novel features herein disclosed. In the following detailed description, numerous specific details are set forth in order to provide a thorough understanding of the present invention. However, it will be understood by those skilled in the art that the present invention may be practiced without these specific details. In other instances, well-known methods, procedures, and components have not been described in detail so as not to obscure the present invention.

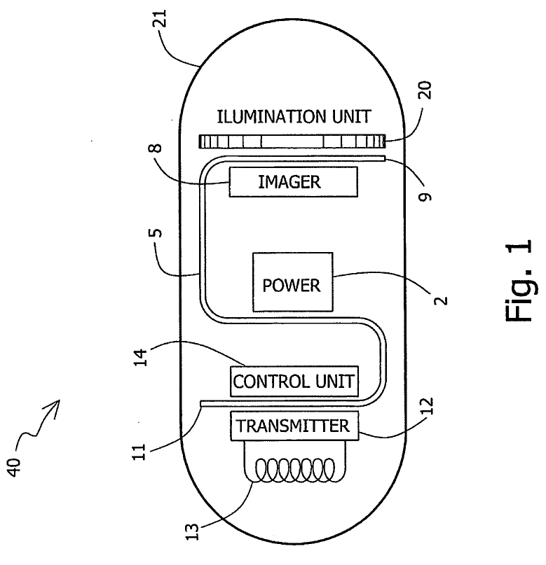

Reference is now made to FIG. 1, which illustrates components of an in-vivo sensing device, for example imaging device 40, according to some embodiments of the present invention. Device 40 typically may be or may include an autonomous swallowable capsule, but device 40 may have other shapes and need not be swallowable or autonomous. Embodiments of device 40 are typically autonomous, and are typically self-contained. For example, device 40 may be a capsule or other unit where all the components are substantially contained within a container or shell, and where device 40 does not require any wires or cables to, for example, receive power from an external source or transmit information. Device 40 may communicate with an external receiving and display system to provide display of data, control, or other functions. For example, power may be provided by an internal battery or a wireless receiving system. Other embodiments may have other configurations and capabilities. For example, components may be distributed over multiple sites or units. Control information may be received from an external source.

Devices according to embodiments of the present invention, including imaging, receiving, processing, storage and/or display units suitable for use with embodiments of the present invention, may be similar to embodiments described in U.S. Pat. No. 5,604,531 to Iddan et al., and/or in co-pending U.S. patent application Ser. No. 09/800,470 entitled A DEVICE AND SYSTEM FOR IN VIVO IMAGING, both of which are assigned to the common assignee of the present invention and which are hereby incorporated by reference. Of course, devices and systems as described herein may have other configurations and other sets of components.

In one embodiment, all of the components may be sealed within the device body (the body or shell may include more than one piece); for example, an imager 8, illumination unit, for example a hybrid illumination unit 20, power units 2, and transmitting 12 and control 14 units, may all be sealed within the device body.

The device 40 is capsule shaped and can operate as an autonomous endoscope for imaging the GI tract. However, other devices, such as devices designed to be incorporated in an endoscope, catheter, stent, needle, etc., may also be used, according to embodiments of the invention.

According to one embodiment of the invention, the various components of the device 40 are disposed on a circuit board 5, for example a flexible circuit board or a circuit board having rigid sections and flexible sections. Such circuit boards may be similar to embodiments described in U.S. application Ser. No. 10/879,054 entitled IN VIVO DEVICE WITH FLEXIBLE CIRCUIT BOARD AND METHOD FOR ASSEMBLY THEREOF, and U.S. application No. 60/298,387 entitled IN VIVO SENSING DEVICE WITH A CIRCUIT BOARD HAVING RIGID SECTIONS AND FLEXIBLE SECTIONS, each incorporated by reference herein in their entirety. Preferably, according to one embodiment the components may be arranged in a stacked vertical fashion. For example, one portion 11 of the circuit board may hold a transmitter 12 and an antenna 13. Another portion 9 of the circuit board may include an illumination unit, for example a rounded hybrid illumination unit 20.

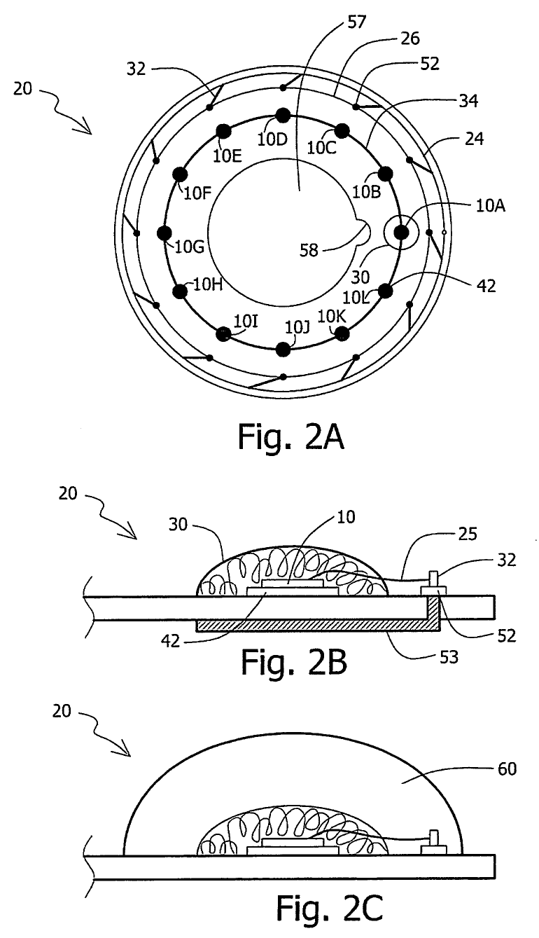

Reference is now made to FIG. 2A showing a schematic view from the top of a hybrid illumination unit 20 in accordance to one embodiment of the present invention. According to one embodiment, the hybrid illuminating unit 20 may include one or more discrete light sources 10A, 10B, to 10L or may include only one light source. The light source(s) 10A, 10B, to 10L of the hybrid illuminating unit 20 may be white light emitting diodes, such as the light sources disclosed in co-pending U.S. patent application Ser. No. 09/800,470 to Glukhovsky et al. However, the light source(s) 10A, 10B, 10L of the hybrid illuminating unit 20 may also be any other suitable light source, known in the art, such as but not limited to monochromatic LEDS, incandescent lamp(s), flash lamp(s) or gas discharge lamp(s), or any other suitable light source(s).

According to some embodiments the hybrid illumination unit 20 may include a printed circuit board (PCB) made of, for example, silicone or plastic. Other suitable materials may be used. According to one embodiment the hybrid illumination unit 20 may be a ring shaped for example with an internal circle e.g. a rounded hole 57 in its center. Typically, the hybrid illumination unit 20 has compatible measurements for a suitable incorporation into an in vivo device 40, for example an in vivo imaging device. The hybrid illumination unit 20 may be of a different shape other than a ring shape e.g. a rectangular or square shape, or of any other form compatible for fitting into an in vivo device.

According to one embodiment of the invention two printed traces 24 and 34, are printed on the hybrid illumination unit 20. Each of the printed traces 24 and 34 may be connected either to the positive terminal of the battery 2, or to the negative terminal of the battery 2 through printed trace 53 (shown in FIG. 2B). According to some embodiments of the invention another printed trace 26, which may be located, for example, between printed trace 24 and 34, may include a plurality of pads 52 for wire bonding, for example a plurality of resistors 32.

According to one embodiment of the present invention, conductive pads 42, for example metal pads for chip bonding may be placed or molded on printed trace 34, to provide connections for a plurality of discrete light sources 10A-10L, for example, to a number of LED chips. Each light source 10A-10L may be associated with one or more additional components such as one or more resistor(s) 32, which may be connected to pad 26. Pad 26 may, for example, enable control over the amount of illumination generated by light source 10A-10L. For example, a processor associated with device 40 may be able to use resistors 32 to generate different intensities of light in different parts of the GI tract, such as, 200 lux of light in the small intestine and 300 lux in the colon. Illumination may be controlled and customized for selected illumination functions. Resistor(s) 32 may be variable or permanent, for example a permanent resistor may enable normalized light output from a plurality of light sources.

According to one embodiment of the present invention, an optical resin 30 may be placed over each light source 10A-10L, for example over each LED chip, providing different spectra of illumination (e.g, red, green or blue spectra, infra-red spectra or UV spectra). Furthermore, in certain embodiment, the various light sources 10A-10L may provide different spectra of illumination (e.g, red, green or blue spectra, infra-red spectra or UV spectra). In such embodiments, the illumination provided can be arranged in such a way that the illumination direction is different for each channel employing a different spectrum.

According to some embodiments, a depression 58, positioned in the internal circle of the illumination unit, serves as a direction marker during the hybrid illumination unit 20 installation within the in vivo device. In an alternate embodiment, depression 58 may be of other suitable shapes.

Reference is now made to FIG. 2B showing a schematic closer view from the side of a light source 10, for example a LED, installed into a hybrid illumination unit 20, in accordance to one embodiment of the present invention.

According to some embodiments the light source 10, may be placed over a conductive pad 42, for example a chip bonding pad, and may be connected through wire 25 to a pad 52, such as a pad for wire bond. According to some embodiments a resistor 32 may be placed on top of pad 52, for example, in order to control the light source 10 illumination intensity or other parameters such as amplitude.

According to one embodiment a plurality of local control units may be suitably connected to the light sources 10A, 10B, to 10L of hybrid illumination unit 20 for controlling the energizing of each light source 10A, 10B, to 10L of the hybrid illumination unit 10 and/or for controlling the energizing of a sub-group of light sources, for example light sources 10A to 10B. According to one embodiment each local control unit may be used for switching one or more of the light sources 10A, 10B, to 10L on or off, or for separately controlling the intensity of the light produced by each light source 10A, 10B, to 10L.

According to one embodiment of the present invention, a conductive pad and/or electrical wire 53, may be placed or molded on the hybrid illumination unit 20, to provide connections for example to battery 2. According to one embodiment of the present invention, directly over the light source 10 an optical resin 30 is placed, intended to form a different spectra of illumination (for example, as was described with reference to FIG. 2A). FIG. 2C depicts a hybrid illumination unit 20 with lens 60, for example a molded optical lens, mounted on it, in accordance with one embodiment of the present invention. According to one embodiment of the present invention, directly over each light source 10 a lens is placed, intended to form different illumination direction to each light source. In one embodiment of the present invention lens 60 may be made for example from a suitable silicon or transparent plastic, such as for example, ABS, polycarbonates or other suitable materials. Lens 60 may be manufactured by, for example, injection molding. The type of lens employed, its shape and position over the hybrid illumination unit 20, may determine the direction of illumination so that different areas of bodily compartments may be illuminated. Thus, the lens may be chosen according to the in vivo device's target area, for example, in vivo devices targeted to image the stomach lumen may require a different lens arrangement than a device targeted to image the esophagus.

According to some embodiments, the hybrid illumination unit 20 may include an amorphous lens, capable of changing its form and focus by way of an electrical current directed towards it, either through remote manual control or automatically as the device travels through the body.

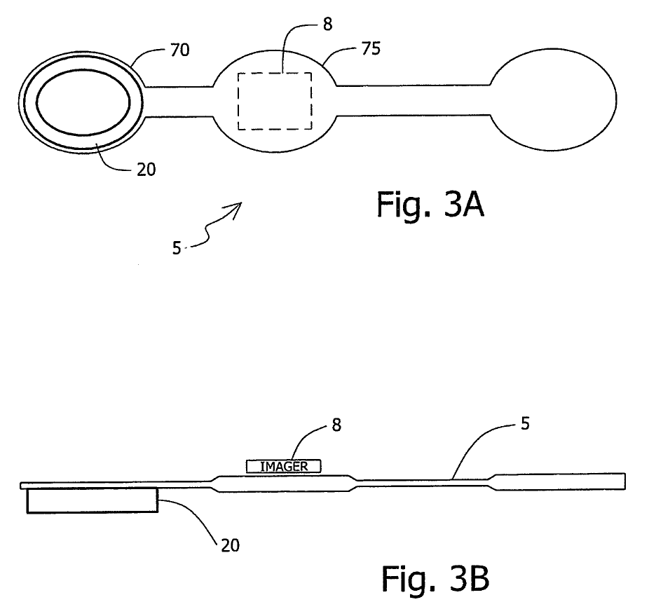

Reference is now made to FIG. 3A showing an example embodiment of a circuit board 5, for example a one sheet flexible circuit board, or a rigid-flex circuit board, in its spread out form, after the hybrid illumination unit 20 has been installed on the circuit board 5 and before it is folded and inserted into an in vivo device, for example, a capsule, according to an embodiment of the invention.

According to one embodiment a portion or section of the circuit board 5 may have a set of components mounted or disposed upon it. According to one embodiment portion 70 of the circuit board 5 may include, for example the hybrid illumination unit 20, whereas portion 75 of the circuit board 5 may include the imager 8. In alternate embodiments, other components layouts, may be arranged on a circuit board with different shapes or on other in vivo device's components and/or installed in other compartments of the in vivo device.

FIG. 3B depicts a side view of the circuit board 5 in its spread form, prior to it's insertion into the in vivo device according to one embodiment of the present invention. In this embodiment the hybrid illumination unit 20 is installed on a bottom portion of the circuit board 5, although the hybrid illumination unit 20 may also be installed in several other areas of the circuit board 5.

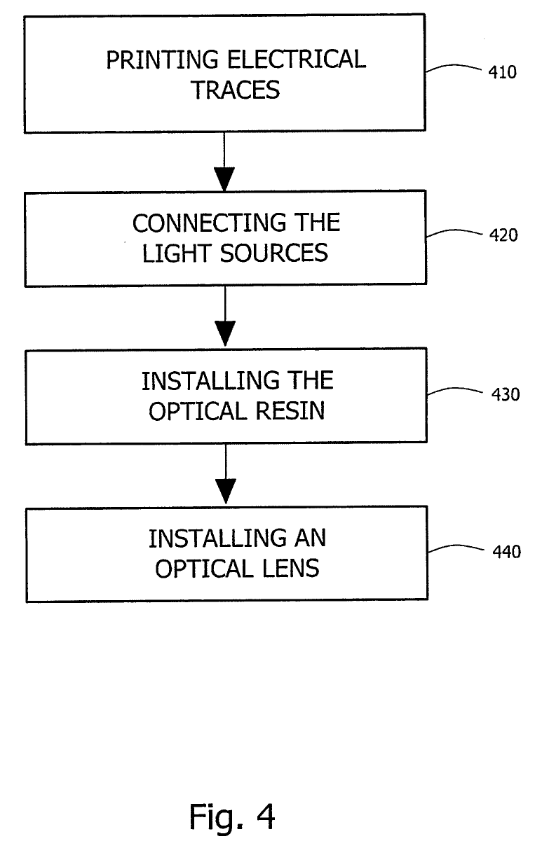

A method for producing an in vivo imaging device, which includes a hybrid illumination unit, according to different embodiments of the invention is depicted in FIG. 4. According to some embodiments of the present invention, step 410 includes printing electrical traces on a substrate, such as a PCB. For example, a first electrical circuit, which may be wired to pads where the light sources 10, may be connected and a second electrical circuit which may be wired to pads where a plurality of resistors may be mounted on a PCB. Step 420 includes connecting the light sources to the resistors in order to be able to achieve different illumination intensities. Step 430 includes installing an optical resin above the light sources in order to create a vast spectrum of illumination inside the body. Different joining methods may be used during the hybrid illumination unit 20 assembly and/or for connecting the hybrid illumination unit 20 to the circuit board 5. For example welding methods e.g. laser welding, spin welding, Herman welding and vibration welding, and/or melt down methods, and/or ultrasonic joining and/or fraction fitting. Step 440 includes installing an optical lens above the hybrid illumination unit in order to direct and focus the illumination.

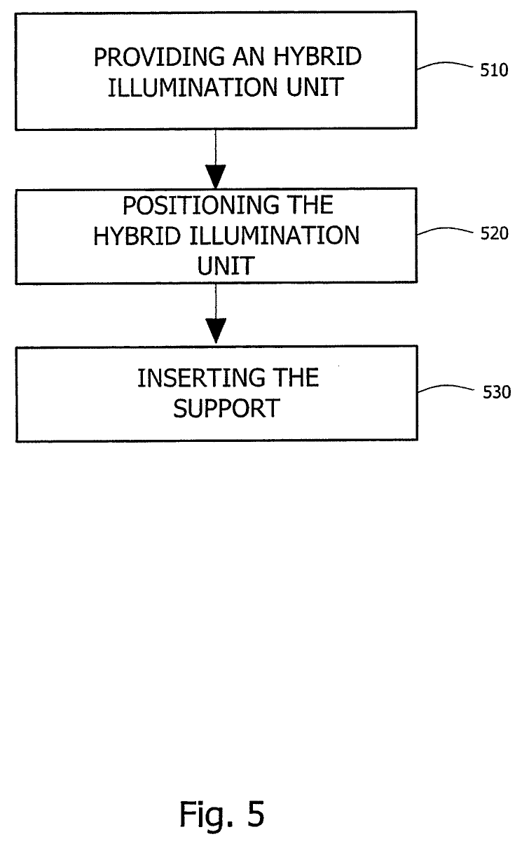

A method for providing in vivo illumination according to another embodiment is shown in FIG. 5. According to one embodiment the method may include providing a hybrid illumination unit (510) and positioning the hybrid illumination unit on a support (520), for example on a flexible PCB and inserting the support into a housing of an in vivo device (530). Other steps or combinations of steps may be used.

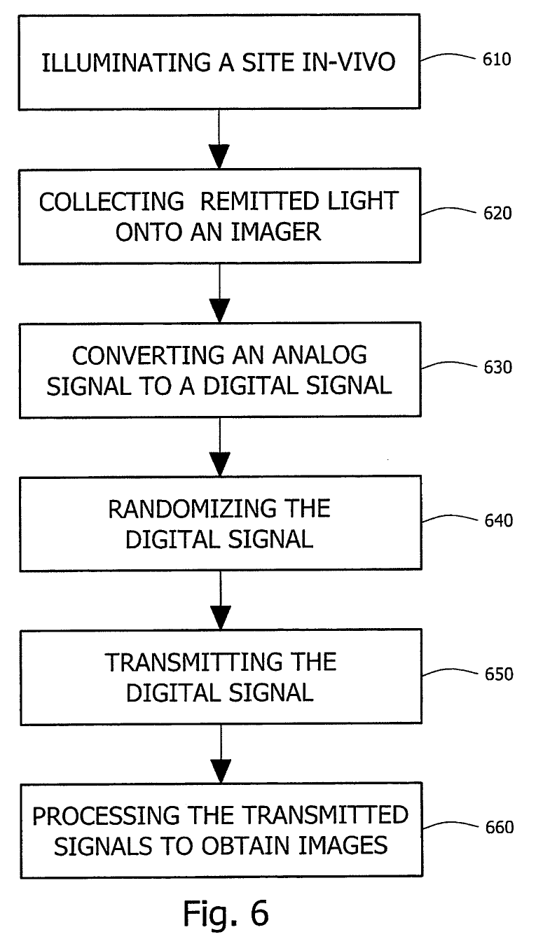

A method for providing in vivo illumination according to some embodiments of the present invention is shown in FIG. 6. The method for in vivo imaging may include the following steps: illuminating a site in vivo (610), for example by using the hybrid illumination unit 20; collecting remitted light onto an imager 8, thereby generating an analog signal (620); converting the analog signal to a digital signal (630); randomizing the digital signal (640); transmitting the digital signal to a receiving system (650) and processing the transmitted signals to obtain images of the in vivo site (660). Other steps or combinations of steps may be used.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

What is claimed is:

1. A device for in vivo imaging comprising an imager, a hybrid illumination unit and a lens located above said hybrid illumination unit.

2. The device according to claim 1, wherein the hybrid illumination unit comprises a plurality of light sources.

3. The device according to claim 2 wherein a lens is mounted separately on each light source.

4. The device according to claim 1 comprising a support, wherein said hybrid illumination unit is positioned on the support.

5. A method for the manufacture of an in vivo sensing device, the method comprising the steps of:
positioning a hybrid illumination unit on a support; and
folding said support into a device housing.

6. The method according to claim 5, comprising providing an imager.

7. The method according to claim 5, comprising providing a transmitting unit.

8. The method according to claim 5, comprising providing a power unit

9. The method according to claim 5, comprising providing a control unit.

10. The method according to claim 5, wherein said support is selected from the group consisting of: a PCB, a flexible circuit board, a rigid-flex circuit board.

11. A method for the manufacture of a hybrid illumination unit, the method comprising the steps of:
printing electrical traces on a substrate,
disposing a light source on said electrical traces; and
installing a lens above the light source.

12. The method according to claim 11 comprising installing an optical resin above said light source.

13. The method according to claim 11, comprising installing a lens above a discrete light source.

14. The method according to claim 11, comprising installing a lens above a plurality of light sources.

15. The method according to claim 11, comprising installing a resistor on said electrical traces.
